(19) United States
(12) Patent Application Publication
(10) Pub. No.: US 2024/0293299 A1
(43) Pub. Date: Sep. 5, 2024

(54) COSMETIC AEROSOL PRODUCT

(71) Applicant: Conopco, Inc., d/b/a UNILEVER, Englewood Cliffs, NJ (US)

(72) Inventors: Merion Evans, Brough (GB); Louise Ombler McMaster, Leeds (GB)

(21) Appl. No.: 18/590,499

(22) Filed: Feb. 28, 2024

(30) Foreign Application Priority Data
Mar. 1, 2023 (EP) 23159327.8

(51) Int. Cl. A61K 8/27; A61K 8/04; A61Q 15/00; B65D 83/14

(52) U.S. Cl. CPC A61K 8/27; A61K 8/046; A61Q 15/00; B65D 83/752; A61K 2800/74; A61K 2800/87

(57) ABSTRACT

A cosmetic aerosol product comprising a pressurised container holding a base composition and a propellant which is a mixture of a compressed gas and a liquefied gas at a weight ratio of from 0.1:99.9 to 10:90; wherein the pressurised container comprises:
i). a pressure release valve;
ii). a reservoir containing the base composition and the propellant; and
iii). a dip tube arranged for the transport of the base composition from the reservoir to the pressure release valve;
   wherein the base composition comprises ethanol and a non-volatile, ethanol-soluble cosmetic active and the weight ratio of the base composition to the propellant is from 30:70 to 90:10.

### FIELD OF INVENTION

The present invention relates to cosmetic aerosol products having a reduced content of liquified propellant, such products being capable of reducing volatile organic carbon (VOC) emissions to the environment.

### BACKGROUND

There are numerous publications describing aerosol products with a reduced content of liquified propellant.
EP 753,561 A1 (P&G, 1997) discloses an aerosol package comprising “ozone friendly” propellants, such as compressed gas or nitrogen, amongst other options.
U.S. Pat. No. 8,191,739 B1 (Randy Cash, 2012) discloses an aerosol dispenser that is pressurised by both compressed gas and liquified gas and which is suitable for foaming products.
WO 2022/012947 A1 (Unilever, 2022) discloses aerosol products having reduced VOC content wherein the propellant comprises an insoluble compressed gas and 5 to 15% by weight of a liquefied gas and wherein the pressure in the container is from 9×10⁵ to 11×10⁵ Pa.
US 2023/278505 A1 (P&G, 2023) discloses spray devices for antiperspirant or deodorant compositions with a compressed gas propellant.

### SUMMARY OF THE INVENTION

It is an object of the present invention to provide a cosmetic aerosol product that uses a relatively low level of liquified gas propellant and yet has good sensory and good delivery properties.

Products according to the present invention have good sustainability credentials: first, because of their low content of liquified propellant gas, which can often act as a greenhouse gas and secondly, because they enable efficient, non-wasteful delivery of cosmetic composition to the surface of the human body. Surprisingly, this is enabled with good to reasonable longevity of spray performance in terms of spray delivery. In addition, the present invention can deliver good sensory properties for the spray when topically applied.

In a first aspect of the invention there is provided a cosmetic aerosol product comprising a pressurised container holding a base composition and a propellant which is a mixture of a compressed gas and a liquefied gas at a weight ratio of from 0.1:99.9 to 10:90; wherein the pressurised container comprises:
i). a pressure release valve;
ii). a reservoir containing all of the base composition and all of the propellant; and
iii). a dip tube arranged for the transport of the base composition from the reservoir to the pressure release valve;

wherein the base composition comprises ethanol and a non-volatile, ethanol-soluble cosmetic active and the weight ratio of the base composition to the propellant is from 30:70 to 90:10.

In a second aspect of the invention, there is provided a method of attaining a cosmetic benefit comprising the topical spray application of base composition and propellant mixture as described in the first aspect of the invention.

In a third aspect of the invention, there is provided a method of manufacture of cosmetic aerosol product according to the first aspect of the invention.

The present invention involves the use of a propellant comprising a liquified gas and compressed gas at a relatively narrow ratio range in combination with an ethanolic base composition comprising a non-volatile, ethanol-soluble cosmetic active, such that the ratio of base to propellent is also selected to be within a relatively narrow range.

Without wishing to be bound by theory, the use of lower amounts of the liquified gas, versus the compressed gas and versus the amount of base, may lead to better delivery of the base composition, including the non-volatile, ethanol-soluble cosmetic active contained therein, as a result of the lower volatility of the combined propellant plus base composition.

### DETAILED DESCRIPTION

The balance between the compressed gas and liquified gas in the propellant is critical to the success of the invention. This ratio is from 0.1:99.9 to 10:90, preferably from 0.5:99.5 to 8:92, still more preferably from 1:99 to 6:94 and most preferably from 1:99 to 4:96.

The balance between the base composition and the propellant is also important to the success of the invention. This ratio is from 30:70 to 90:10, preferably from 40:60 to 90:10, more preferably from 45:55 to 90:10 and most preferably from 50:50 to 90:10. These preferred ratios help the environmental credentials of compositions of the invention and also the delivery and/or sensory benefits attained.

When it is desired to include a vapour phase tap (VPT) in the pressure release valve (vide infra), it is desirable to reduce the highest preferred level for the base to propellant ratio. Hence, in embodiments comprising a VPT, the base to propellant ratio is preferably from 30:70 to 80:20, more preferably from 40:60 to 80:20, and most preferably from 45:55 to 80:20. Reasons for these preferences are as detailed in the paragraph immediately above and also relate to spray quality.

Herein, “non-volatile”, particularly with regard to ethanol-soluble cosmetic actives, should be understood to refer to materials having a vapour pressure of less than 2 mm Hg (267 Pa) at 20° C.; preferably less than less than 0.2 mm Hg (26.7 Pa) at 20° C. and more preferably less than 0.02 mm Hg (2.67 Pa) at 20° C. In particularly preferred embodiments, these criteria should be understood to exclude conventional fragrances and their constituents.

Herein, topical application refers to application to the skin of the human body, in particular to the underarm regions of the human body.

Herein, the term “spray quality” refers to the extent of droplet breakup achieved on use of the aerosol products. It is desirably to achieve relatively high droplet breakup, such that the spray contains less than 10% of droplets greater than 150 microns in diameter and preferably less than 5% of such droplets.

Herein, methods and uses should be understood to be cosmetic methods/uses, i.e. non-therapeutic methods/uses. Likewise, compositions of the invention are cosmetic compositions, i.e. non-therapeutic.

Herein, all percentages, parts, and ratios are by weight, unless otherwise indicated.

Herein, references to an amount of a material or materials refer to the total amount of material(s) of the type indicated.

Herein, references to an amount of a material are with reference to the total composition of which it is a part, unless otherwise indicated.

Herein, “application” and “applied” relate to application to the skin of the human body, in particular the underarm regions, unless the context dictates otherwise.

Herein, features expressed as “preferred” with regard to a particular aspect of the invention should be understood to be preferred with regard to each aspect of the invention.

Herein, preferred, particularly preferred and especially preferred features of the invention are particularly preferred when used in combination with other preferred, particularly preferred, and especially preferred features of the invention.

Herein, “ambient conditions” refer to 20° C. and 1 atmosphere pressure, unless otherwise indicated.

Herein, indicated pressures should be understood to be “gauge” pressures, i.e. pressures above atmospheric pressure, with the exception of vapour pressures.

Herein, the word “comprising” is intended to mean “including” but not necessarily “consisting of” or “composed of.” In other words, the listed steps or options need not be exhaustive.

Herein, except in the examples or where otherwise explicitly indicated, all numbers in this description and claims indicating amounts of material, physical properties of materials and/or use are to be understood as modified by the word “about”.

Herein, numerical ranges expressed in the format “from x to y” are understood to include x and y. When for a specific feature multiple preferred ranges are described in the format “from x to y”, it is understood that all ranges combining the different endpoints are also contemplated.

Herein, the base composition should be understood to refer to all components of the composition other than the propellant.

The base composition comprises ethanol as an essential component. The content of ethanol in the base composition is preferably at least 50%, more preferably at least 60% and most preferably at least 75%.

In some embodiments, the ethanol content of the base composition is preferably from 75 to 98%, more preferably from 80 to 97.5% and most preferably from 90 to 97.5%.

Having the preferred levels of ethanol in the base composition as indicated in the two paragraphs immediately above helps with homogeneity of the base composition and the spray quality delivered when products according to the invention are used.

In preferred embodiments the base composition is low in water content, preferably containing less than 15%, preferably less 6% water and more preferably less than 0.5% water. This aids homogeneity of the composition.

In preferred embodiments the base composition is not physically separated from the propellant or any component thereof. In this way, the propellant can act directly upon the base composition and may achieve better spray quality and/or delivery.

The cosmetic active is non-volatile and ethanol-soluble. The non-volatility aids the efficient delivery of the cosmetic active to its intended target, which is typically the surface of the human body. Evaporation of the cosmetic active during its spray application reduces efficiency of delivery, hence the requirement for non-volatility.

The ethanol-solubility of the cosmetic active is required for its compatibility with the ethanol in the base composition. It is highly preferred that the ethanolic base composition is homogeneous; hence, it is preferred that the cosmetic active is fully solubilised by the ethanol in the base composition. Having a homogeneous base composition enhances spray quality and avoids possible valve blockage issues. It can also help with spray capture efficiency and/or sensory benefits.

Subject to the criteria indicated above, the cosmetic active may be any of those known in the art that is suitable for spray application. Preferred cosmetic actives include deodorant actives, i.e. actives that can reduces body odour or at least the perception thereof. Preferred deodorant actives are organic in nature, to aid with their ethanol solubility.

Herein, “organic” deodorant actives should be understood to refer to actives comprising carbon and hydrogen. Such actives may also comprise other elements, including metals. Herein, deodorant actives comprising carbon, hydrogen and metal are termed organometallic deodorant actives.

Suitable organic deodorant actives for use in the present invention include 2-methyl-5-cyclohexylpentanol (SymDeo B125), butyloctanoic acid, dipropylene glycol, DTPA, ethylhexyl glycerol (Sensiva SC50), glyceryl laurate, lactic acid, menthyl lactate (Freshcolat ML), Octenidine hydrochloride (TPI 2007; Sensidin DO); pentylene glycol, piroctone olamine (Octopirox); poly(hexamethylenebiguanide) hydrochloride (Cosmocil CQ); polyquaternium-6 (Merquat 100); propanediol; propylene glycol; triclosan; triethyl citrate (Hydragen CAT); zinc neodecanoate; zinc phenol sulphonate; and zinc ricinoleate.

Organometallic deodorant actives are a preferred group of cosmetic actives for use in the invention since they typically have low volatility and good ethanol solubility. They can also have extremely good efficacy when delivered in accordance with the invention. Amongst such actives, zinc carboxylates salts are particularly preferred, these actives typically being zinc dicarboxylates, reflecting the stoichiometry of salts formed between divalent zinc ions and monovalent carboxylates. Herein, references to zinc carboxylates also include zinc dicarboxylates.

Preferred zinc carboxylates salts are salts of a fatty acid having from 6 to 18 carbon atoms. Such salts are relative hydrophobic in nature, enhancing the ease with which they can be formulated with ethanolic base compositions and the liquified gas propellant, which is usually hydrophobic in nature.

Preferred zinc carboxylate salts are salts of a fatty acid having from 8 to 14 carbon atoms, particularly 8 to 12 carbon atoms.

Preferred zinc carboxylate salts are salts of a saturated fatty acid, in particular salts of a saturated branched chain fatty acid having from 8 to 12 carbon atoms and more.

Other suitable deodorant actives for use include polyquaternium compounds, such as those disclosed in WO 16/012417 A1 (Beiersdorf, 2016) and WO 16/012420 A1 (Beiersdorf, 2016).

When employed, preferred PQ compounds may be selected from a list consisting of PQ-5, PQ-6, PQ-7, PQ-10, PQ-11, PQ-16, PQ-22, PQ-28, PQ-37 and mixtures thereof. Particularly preferred PQ compounds are PQ-6, PQ-16, PQ-22 and PQ-37; especially preferred are PQ-6, PQ-16 and PQ-22 and most preferred is PQ-6.

Herein, PQ-6, i.e. polyquaternium-6, is polymeric quaternary ammonium salt of diallyldimethylammonium chloride.

The cosmetic active is typically included in the base composition at a level of from 0.01 to 10%, preferably at from 0.1% to 7.5% and most preferably at from 0.2% to 5%. These levels are of particular relevance when the cosmetic active is a deodorant active.

A preferred additional component in compositions used in the invention is a fragrance or fragrance oil, sometimes alternatively called a perfume (oil). The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to.

The fragrance may be present “free”, herein simply termed “fragrance” and/or it may be encapsulated by methods known in the art, in order to delay its release. Often, the delayed release of the fragrance is triggered by contact with moisture or, preferably, by friction. In preferred embodiments, compositions of the invention comprise both free and encapsulated fragrance.

The amount of fragrance oil in the total composition is preferably up to 5%, advantageously is at least 0.5% and particularly from 0.8% to 3.5%.

The amount of fragrance oil in the base composition is preferably up to 20%, advantageously is at least 1% and particularly from 2% to 8%.

The propellant is a mixture of a compressed gas and a liquefied gas at a weight ratio of from 0.1:99.9 to 10:90.

Preferred compressed gases are inert, i.e. unreactive materials. A particularly preferred inert gas is nitrogen. Other suitable inert gases are carbon dioxide, helium and argon; although these gases are less preferred for practical reasons, in particular their greater expense.

Prior to first use, the pressure exerted by the total propellant within products according to the invention is preferably from 85 to 125 psig (0.59 to 0.86 MPa) at 25° C., these pressures assisting with good spray quality and delivery.

Prior to first use, the pressure exerted by the compressed gas component of the propellant used in the present invention is preferably from 0.10 to 0.40 MPa at 25° C., these pressures assisting with the duration of good spray quality and delivery.

Suitable liquified gas propellants for use herein include hydrocarbons, in particular C3-C4 hydrocarbons, and/or hydrofluorocarbons (otherwise known as HFCs), in particular C2-C4 HFCs, and dimethyl ether.

Preferred liquified gas propellants are blends of propane, butane and/or isobutane, with the less preferred addition of an HFC.

Commercially acceptable liquified gas propellants are AP21, AP22 and AP40. AP21 is a hydrocarbon blend of butane, isobutane and propane (70:29:1). AP22 is typically a blend of n-butane (ca. 30%), isobutane (ca. 67%) and minor amounts of other hydrocarbons, including propane. AP40 is a blend of propane, n-butane and isobutane, typically at a ratio of about 22:24:54.

When employed, preferred HFCs have from 2 to 4 carbon atoms. A commercially acceptable HFC is HFC-152a, otherwise known as R152a and having the chemical name 1,1-difluoroethane (DFE). However, it is preferred that compositions according to the invention are free of HFC in order to minimise greenhouse gas content.

Other HFCs that may be employed are hydrofluoroolefins (HFOs), such as 1,3,3,3-tetrafluoropropene, otherwise known as HFO-1234ze.

Essential components of hardware used in products according to the invention are:
i). a pressure release valve;
ii). a reservoir containing all of the base composition and all of the propellant; and
iii). a dip tube arranged for the transport of the base composition from the reservoir to the pressure release valve.

The pressurised container holds the aerosol composition under pressure and the pressure release valve releases the composition and allows it to form an aerosol on exit from the container.

The reservoir containing all of the base composition and all of the propellant, i.e. the compressed gas and a liquefied gas, is a single reservoir. The liquefied gas typically equilibrates between the base composition and a gaseous space above the base composition. The compressed gas is typically insoluble in the base composition and sits in the aforementioned gaseous space above the composition. The combination of the two types of propellant in the same reservoir as the base composition enables optimum maintenance of spray capture on first activation. Further, as the contents of the container deplete with repeated use (in particular the propellant which depletes faster than the base), maintenance of good spray rate and delivery is enabled by having the two types of propellant in the same reservoir as the base composition.

Other features typically present within spray devices may also be employed, including:
a valve cup for holding the valve firmly within an outer body of the pressurised container, often used in combination with a valve cup gasket;
a push button for “activating” the pressure release valve; i.e., for opening the valve;
A swirl chamber for enhancing the quality of the spray exiting the pressure release valve. These are sometimes known as “inserts”.

The pressure release valve generally has a valve stem, a body and a spring to aid with closure following activation. A gasket to seal the valve within an associated valve cup is also generally employed. Preferred valve stem orifices have a diameter of from 0.2 to 0.6 mm and more preferably from 0.35 to 0.45 mm.

Other preferred features in the pressure release valve include a vapour phase tap (VPT) and a restricted tail piece (RTP). The VPT enhances the mixing of the propellant and the base composition and helps with spray quality and control of spray rate. Preferred VPTs have a diameter of 0.30 to 0.50 mm, more preferably 0.35 to 0.45 mm.

Herein, references to the “diameter” of a component, such as VPT, RTP or dip tube, refer to its internal diameter.

In some embodiments, it is preferred that the pressure release valve does not have a VPT. Such embodiments typically have a high base to propellant ratio, such as from 75:25 to 90:10 or from 80:20 to 90:10. The reason for this preference is maintenance of spray quality.

Preferred spray rates for products of the invention are from 0.1 to 0.8 g/s, preferably from 0.2 to 0.6 g/s.

Herein, spray rates are initial spray rates, i.e. spray rates on first actuation of the can and before its contents are significantly depleted.

The RTP helps control the rate of entry of the base composition into the valve; therefore it is also involved in spray quality and spray rate control. Preferably the RTP has a diameter of from 0.5 to 1.0 mm, more preferably from 0.6 to 0.7 mm.

In a preferred aspect of manufacture of products according to the invention, the base composition is prepared first, typically using high shear. The base composition is then placed in the pressurised can and the compressed gas is added. Finally, the liquified gas is added. A benefit of adding the liquified gas after the compressed gas is that the liquified gas helps to wash back any base composition partially forced out of the can by the temporary pressure release when starting to add the liquified gas. In addition, the pressure build on adding the liquified gas after the compressed gas is much quicker than vice versa.

### EXAMPLES

The following Examples are illustrative of the invention claimed and are non-limiting.

Example 1 and Comparative Example 1 detailed in Table 1 were prepared as described hereinbelow. Quantities of components are indicated as parts by weight.

TABLE 1

| | | Comparative | |
|---|---|---|---|
| | Base | Example 1 | Example 1 |
| DRM | 2.5 | 1.0 | 1.38 |
| Fragrance | 4 | 1.6 | 2.21 |
| EtOH | 93.5 | 37.4 | 51.76 |
| Liquified gas | — | 60.0 | 44.01 |
| Nitrogen | — | — | 0.63 |

The same base formulation, as indicated in Table 1, was used for both Example 1 and Comparative Example 1. The deodorant raw material (DRM) used was a mixture of zinc neodecanoate, isopropyl myristate and fragrance at a ratio of 4:1:0.01, respectively.

In Comparative Example 1, the base formulation was gassed with a conventional propellant blend consisting of HFC-152a and AP22 (at a ratio of 47:53). The base to propellant ratio used was 40:60.

In Example 1, the base formulation was first gassed with nitrogen to a pressure of ca. 27 psig (0.19 MPa) and then the liquified gas propellant was added. The liquified gas used was AP40. The amount of nitrogen added was calculated to be 0.63% of the total composition.

The same conventional spray cans were used to spray both Example 1 and Comparative Example 1. The spray cans had a 210 ml brimful capacity and were equipped with a 0.3 mm valve stem, a 0.41 mm VPT and a 0.64 mm RTP. The sprays were held vertically and sprayed onto filter papers from a distance of 10 cm.

Duplicate samples of Example 1 and Comparative Example 1 were tested, 6 times each. Spray capture of the base (i.e. allowing for the somewhat different base to propellant ratios of the products) was calculated on spraying the cans from full and then again when the can was only 50% full. The mean results are presented in Table 2.

TABLE 2

| | | Mean base | Mean | Mean |
|---|---|---|---|---|
| Example | % Fill on can | dose (g) | Capture (g) | Capture (%) |
| Comparative 1 | 100 | 0.524 | 0.13 | 24.8 |
| Comparative 1 | 100 | 0.496 | 0.12 | 24.2 |
| Example 1 | 100 | 0.504 | 0.16 | 31.7 |
| Example 1 | 100 | 0.504 | 0.15 | 29.8 |
| Comparative 1 | 50 | 0.500 | 0.15 | 30.0 |
| Comparative 1 | 50 | 0.476 | 0.13 | 27.3 |
| Example 1 | 50 | 0.384 | 0.12 | 31.1 |
| Example 1 | 50 | 0.330 | 0.10 | 30.3 |

The spray captures from Example 1, at 100% fill, were higher than those of Comparative Example 1. At 50% fill, the spray captures from Example 1 were still comparable with those of Comparative Example 1. This is an encouraging result from an environmental perspective, bearing in the mind the reduced liquified gas content of Example 1 (44%) vs. Comparative Example 1 (60%).

The good spray captures for Example 1 equates to a good spray capture for the non-volatile, ethanol-soluble cosmetic active present in the composition, i.e. the zinc neodecanoate.

Table 3 gives details of Example 2, a further composition according to the invention. This composition was prepared in an analogous manner to Example 1 and performs in a similar manner.

TABLE 3

| Ingredient | Amount (%) |
|---|---|
| Ethanol | 51.11 |
| DRM* | 2.80 |
| Fragrance | 2.10 |
| Nitrogen | 0.87 |
| AP40 propellant | 43.12 |

It may be seen that Example 2 had 1.98% nitrogen in the total propellant and a base to propellant ratio of 56:44.

1. A cosmetic aerosol product comprising a pressurised container holding a base composition and a propellant which is a mixture of a compressed gas and a liquefied gas at a weight ratio of from 0.1:99.9 to 10:90; wherein the pressurised container comprises:
i). a pressure release valve;
ii). a reservoir containing all of the base composition and all of the propellant; and
iii). a dip tube arranged for the transport of the base composition from the reservoir to the pressure release valve;
wherein the base composition comprises ethanol and a non-volatile, ethanol-soluble cosmetic active and the weight ratio of the base composition to the propellant is from 30:70 to 90:10.

2. A cosmetic product according to claim 1, comprising fragrance at a level of from 1 to 50% by weight of the base composition.

3. A cosmetic product according to claim 1, wherein the pressure in the container, prior to first use, is from 85 to 125 psig (0.59 to 0.86 MPa) at 25° C.

4. A cosmetic product accordingly to claim 1, wherein the weight ratio of the base composition to the propellant is from 50:50 to 90:10.

5. A cosmetic product accordingly to claim 1, wherein the compressed gas is nitrogen.

6. A cosmetic product accordingly to claim 1, wherein liquified gas is selected from C3-C4 hydrocarbons and/or C2-C4 hydrofluorocarbons.

7. A cosmetic product accordingly to claim 1, wherein the pressure release valve comprises an RTP of from 0.50 to 1.0 mm and a VTP of from 0.30 to 0.50 mm.

8. A cosmetic product accordingly to claim 1, wherein the cosmetic active is a deodorant active.

9. A cosmetic product accordingly to claim 8, wherein the deodorant active is organometallic.

10. A cosmetic product accordingly to claim 8, wherein the deodorant active is a zinc carboxylate.

11. A cosmetic product accordingly to claim 9, wherein the deodorant active is zinc neodecanoate.

12. A cosmetic product according to claim 9, wherein, prior to first use, the pressure exerted by the compressed gas component of the propellant is from 0.15 to 0.40 MPa at 25° C.

13. A method of delivering a cosmetic composition to the surface of the human body comprising the use of a product according to claim 1.

14. A method according to claim 13, wherein the cosmetic active is a deodorant active, and the surface of the human body to which the composition is to be applied is the axilla.

15. A method of manufacture of a cosmetic product according to claim 1, wherein the base composition comprising the ethanol and non-volatile, ethanol-soluble cosmetic active is prepared first; the base composition is then placed in the pressurised container and the compressed gas is added; next, the liquified gas is added and then the container is sealed.
